(19) United States
(12) Patent Application Publication
(10) Pub. No.: US 2016/0256714 A1
(43) Pub. Date: Sep. 8, 2016

(54) VISUALIZATION OF BEAM TRAJECTORIES IN RADIATION THERAPY

(71) Applicant: EMPIRE TECHNOLOGY DEVELOPMENT LLC, Wilmington, DE (US)

(72) Inventors: Leslie Field, Portola Valley, CA (US); Mateusz Bryning, San Jose, CA (US)

(21) Appl. No.: 15/024,888

(22) PCT Filed: Oct. 1, 2013
(86) PCT No.: PCT/US13/62869

§ 371 Date: Mar. 25, 2016

(51) Int. Cl. A61N 5/10

(52) U.S. Cl. CPC A61N 5/1067; A61N 5/1039; A61N 5/1075; A61N 5/1049; A61N 2005/1055; A61N 2005/1058; A61N 2005/1061

(57) ABSTRACT

Technologies are generally described for providing medical imaging during radiation therapy. In one embodiment, a radiation therapy system includes an x-ray apparatus and a monitoring apparatus. The x-ray apparatus is configured for delivery of a therapeutic beam of radiation to tissue of a patient. The monitoring apparatus is configured for monitoring production of free radicals within the tissue during the delivery in order to determine the path taken by the x-ray beam of radiation through the tissue. A computer program product for controlling radiation therapy is provided. Methods for calibrating the radiation therapy system and for providing radiation therapy are included. In some embodiments, the monitoring apparatus may be configured for monitoring production of biomarkers within the tissue, the biomarkers including at least one of free radicals, burst vesicles, dissociation of an adduct and an indication of protein damage.

### CLAIM TO PRIORITY

This application claims priority to PCT/US 13/62869, filed Oct. 1, 2013, which is hereby incorporated by reference in its entirety.

### TECHNICAL FIELD

This disclosure relates generally to medical imaging, and more particularly to medical imaging during radiation therapy.

### BACKGROUND

In radiation therapy, a malignant tumor, often located deep inside the body, is subjected to high doses of ionizing radiation that are lethal to the tumor cells. The irradiation is achieved by use of narrow or focused beams that are targeted at the tumor, and applied in such a way as to minimize damage of the surrounding tissue, while delivering a lethal dose to the tumor. In the procedure, precise targeting of the radiation beam and alignment of the patient are of primary importance. Currently, this alignment is typically done via external alignment marks on the patient, or by physical immobilization of the patient. However, internal organs may involuntarily move during the course of a treatment session, causing misalignment.

Existing methods to ensure proper alignment of the tumor to the radiation beam(s) are inadequate, especially while also keeping track of, and minimizing, radiation exposure within the surrounding healthy tissue. An ideal system would permit the therapist to obtain realtime information about the size, shape, and location of the tumor, the path travelled by the radiation beam, tissue volumes previously exposed to radiation, the level of exposure to each tissue, and the relative locations of all critical organs.

Medical professionals may benefit from enhanced methods and apparatus that make use of imaging techniques to visualize the path of a radiation beam within the body, creating short-term and long-term latent images of the beam trajectory, identifying the tumor, and noting changes in beam trajectory in relation to the tumor and organs.

Unless otherwise indicated herein, the materials described in this section are not prior art to the claims in this application and are not admitted to be prior art by inclusion in this section.

### SUMMARY

Technologies are generally described for providing medical imaging during radiation therapy. In one embodiment, a radiation therapy system includes an x-ray apparatus and a monitoring apparatus. The x-ray apparatus is configured for delivery of a therapeutic beam of radiation to tissue of a patient. The monitoring apparatus is configured for monitoring production of free radicals within the tissue during the delivery in order to determine the path taken by the x-ray beam of radiation through the tissue. A computer program product is provided for controlling radiation therapy. Methods for calibrating the radiation therapy system and for providing radiation therapy are included. In some embodiments, the monitoring apparatus may be configured for monitoring production of biomarkers within the tissue, the biomarkers including at least one of free radicals, burst vesicles, dissociation of an adduct and an indication of protein damage.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a perspective view illustrating a prior art radiation therapy system.

FIGS. 2A-2B, collectively referred to herein as FIG. 2, show perspective views of an illustrative radiation therapy system according to an illustrative example of this disclosure.

FIGS. 3A-3B, collectively referred to herein as FIG. 3, are perspective views illustrating embodiments of a radiation therapy system according to the teachings herein.

FIGS. 4A-4D, collectively referred to herein as FIG. 4, illustrate a cutaway schematic view of a patient, wherein movement of organs relative to a trajectory of a therapeutic beam is depicted.

FIGS. 5A-5D, collectively referred to herein as FIG. 5, are schematic views illustrating the bursting of vesicles.

FIGS. 6A-6D, collectively referred to herein as FIG. 6, are schematic views illustrating an adduct that includes a solubilizing agent and a contrast agent.

FIG. 7 is an exemplary comparative graphic depicting imaging results.

FIG. 8 is a flow chart depicting an exemplary procedure for performing radiation therapy.

FIG. 9 is a flow chart depicting an exemplary procedure for calibrating a radiation therapy system as disclosed herein; all of the figures arranged according to at least some embodiments presented herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Briefly stated, technologies are described for performing medical imaging during radiation therapy. In one embodiment, a radiation therapy system includes an x-ray apparatus and a monitoring apparatus. The x-ray apparatus is configured for delivery of a therapeutic beam of radiation to tissue of a patient. The monitoring apparatus configured for monitoring production of free radicals within the tissue during the delivery in order to determine the path taken by the x-ray beam of radiation through the tissue. A computer program product is included for controlling radiation therapy. Methods for calibrating the radiation therapy system and for providing radiation therapy are included. In some embodiments, the monitoring apparatus may be configured for monitoring production of biomarkers within the tissue, the biomarkers including at least one of free radicals, burst vesicles, dissociation of an adduct and an indication of protein damage. In describing more fully this disclosure, reference is made to the accompanying drawings, in which illustrative embodiments of the present disclosure are shown. This disclosure may, however, be embodied in a variety of different forms and should not be construed as so limited.

Many forms of radiation therapy are known for applying multiple pulses of ionizing radiation to a target tissue site. The radiation is frequently applied in the form of multiple irradiations with narrow or focused radiation beams, so as to minimize exposure of non-target tissues. Success of the therapy strongly depends on delivering a lethal dose to the entire malignancy and minimizing damage to surrounding tissue.

In order to minimize unwanted damage while maintaining therapeutic effect, a therapy plan is generally generated prior to treatment. The plan details the optimal beam shape and direction. Typically, a computed tomography (CT) image or other static volumetric image is taken of the tumor and surrounding tissue. A computerized planning system then determines the contours of the target volume, healthy surrounding tissue, and organs and other sensitive areas at risk of being damaged. The contour data is then used to plan an optimal treatment plan which details the distribution of the radiation, radiation beam direction and shape, and so on.

In addition, prior to radiation treatment, an image of the target volume is taken to align the target volume position to the radiation therapy coordinate system and to verify the accuracy of the plan. The image may be taken by x-ray, fluoroscopy, and in other ways.

However, the target volume may change prior to and/or during treatment such as due to movement of the patient, involuntary shifting of internal organs, and other targeting concerns. It is thus desirable to know, preferably in real time and with high resolution: the exact location, size, and shape of the tumor; the relative locations of all critical organs; the trajectory of the radiation beam in relation to all types of tissues and critical organs; and the radiation dose applied to each volume unit of tissue.

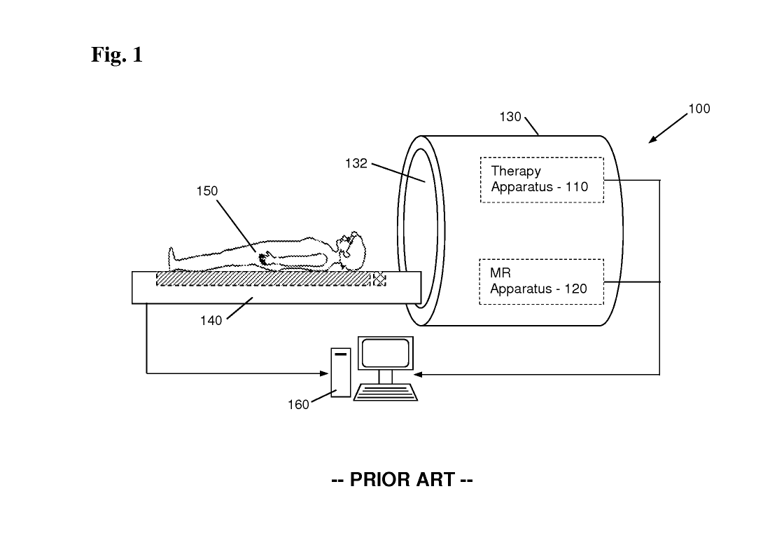

One way of improving delivery of a radiation therapy is by combining a therapy apparatus with an MR apparatus as shown in FIG. 1. FIG. 1 shows a radiation therapy system 100 including a gantry 130 and a patient couch 140. The gantry includes a therapy apparatus 110 and an MR apparatus 120. The therapy apparatus 110 includes a main magnet, like a bore magnet (not shown) and a radiation source such as x-ray, particle beams, etc. The magnet includes a magnet radiation translucent region (not shown) which allows the radiation beams to pass through the magnet. The radiation source is mounted on a rail system (not shown) that allows the radiation source to be rotated about the translucent regions of the magnet. A collimator (not shown) is used to shape the beam of radiation to localize the treatment to a target area. A controller 160 is used to control the therapy apparatus 110.

The MR apparatus 120 includes a radio-frequency (RF) coil assembly (not shown). The RF coil assembly generates radio frequency pulses for exciting magnetic resonance in aligned dipoles of the subject. The RF coil assembly also detects magnetic resonance signals emanating from the imaging region. A controller 160 is used to control the MR apparatus 120.

A patient 150 lying down on the top of the patient couch 140 is placed inside an examination region 132 of the gantry 130. The therapy apparatus 110 provides a radiation beam for therapy treatment. The MR apparatus 120 applies selected magnetic field gradient pulses across the imaging region appropriate to a selected magnetic imaging or spectroscopy sequence. The data received from the MR apparatus 120 is processed by controller 160. The magnetic resonance data controller 160 can perform various functions known in the art. These include image reconstruction, magnetic resonance spectroscopy, etc. Reconstructed magnetic resonance images, spectroscopy readouts, and other processed MR data may be stored in memory (not shown) of the controller 160 and displayed on a graphical user interface.

Prior to the treatment, a plan for the treatment is developed and finalized. The amount of radiation used in radiotherapy is determined depending on tumor type, size, stage, etc. The radiation beam trajectories and geometries are determined from 3-D images generated using the MR apparatus. The current target volume positions are aligned to the coordinate system of the radiation source assembly. Surgical implanted markers and/or other indicators may also be used for alignment. After treatment, the actual dose delivered to each voxel of the target and non-target volumes is determined based on pre-treatment and post-treatment images.

When a beam of ionizing radiation passes through the body, it effects a number of changes on tissues. Notably, the radiation beam produces free radicals within the tissue through bombardment of free water, removing electrons and generating hydronium (H₂0+) cations, which quickly decay to other highly reactive free radicals. The free radicals undergo a multitude of transformations over time, and eventually settle to a stable form, most often a neutral free radical resulting from the breakage of a covalent bond. In solids, these free radicals can be observed at room temperature and are sometimes quite stable, exhibiting no noticeable decay for months or even years.

Free radicals may be observed in solids (e.g., aniline) using methods such as such as ESR (Electron spin resonance), or EPR (Electron Paramagnetic Resonance). These methods are somewhat analogous to nuclear magnetic resonance (NMR), but focus on the resonance of free electrons instead of nuclei. Of particular relevance, ESR is used as a very sensitive method of radiation dosimetry, by monitoring radiation-induced free radicals in solid aniline.

However, ESR and EPR are of limited utility for measuring free radicals in-vivo because of strong absorption by water of the relevant ESR/EPR resonance frequencies. Because of the resulting poor signal penetration, the use of EPRI (the imaging version of EPR) for medical purposes is currently limited to small animal studies.

A double-resonance method based on an effect referred to as the “Overhauser Effect” may be used to overcome the poor signal penetration of ESR/EPR. As a basic overview of the method, consider a solid sample. The sample is placed under a magnetic field. The magnetic field aligns in one direction the spins of all protons in the sample (as in traditional NMR), as well as all electrons in the system. An RF pulse of appropriate frequency is applied to stimulate a resonance in free electrons, as in ESR and EPR above. However, instead of detecting the resonance signal from the electron, as is done in ESR, and which is strongly attenuated by water, a secondary resonance effect (i.e., the Overhauser Effect) is observed instead. In the Overhauser Effect, the resonating electron creates a resonance in a nearby proton (thus the name double-resonance), whose signature resonant frequency can then be monitored at traditional NMR frequencies. The NMR signal of the resonating proton is not attenuated by water, and thus is readily measurable inside living tissue.

Having thus introduced background on radiation imaging, we now turn to features that are provided by this disclosure.

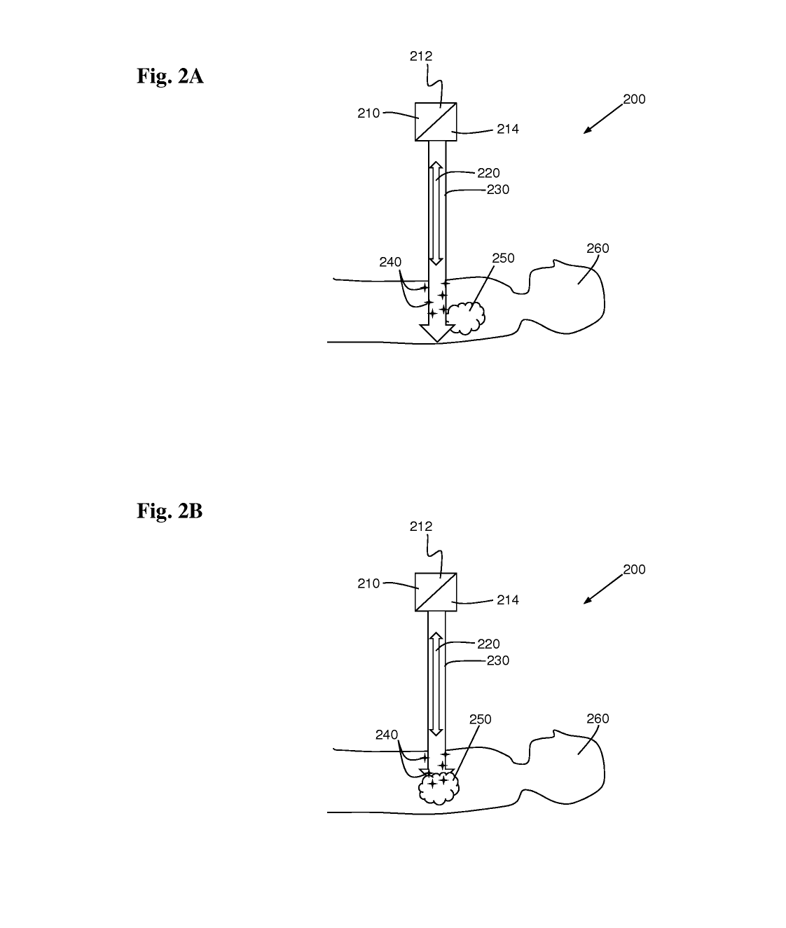

FIGS. 2A and 2B show a radiation therapy system 200 during an x-ray radiation treatment according to one illustrative example of this disclosure. FIG. 2A shows the radiation therapy system 200 earlier in the period of treatment while FIG. 2B shows the radiation therapy system 200 later in that treatment period. The radiation therapy system 200 includes an x-ray apparatus 212 and a monitoring apparatus 214 that employs a technique described in detail below known as Proton-Electron Double-Resonance imaging or PEDRI. The x-ray apparatus 212 and the PEDRI monitoring apparatus 214 are shown in a gantry 210.

The x-ray apparatus 212 is configured for delivery of a therapeutic beam 230 of x-ray radiation to tissue of a patient 260. More specifically, and as previously indicated, the x-ray radiation is illustratively targeted at a tumor shown in FIGS. 2A, 2B as tumor 250. Alternatively, the radiation may be targeted at any other unhealthy tissue. Importantly, the radiation should be applied in such a way as to minimize damage of the surrounding tissue, while delivering a lethal dose to the tumor.

As previously described, when a beam of ionizing radiation, such as an x-ray passes through the body, it effects a number of changes on tissues. Notably, the radiation beam 230 produces free radicals 240 within the tissue. As described below, there are other changes that a beam 230 of radiation may effect which may also be detected. FIGS. 2A, 2B show the path of the x-ray beam 230 having generated free radicals 240. Advantageously, the PEDRI monitoring apparatus 214 of this disclosure is configured for monitoring the production of the free radicals 240 within the tissue of the patient 260 during treatment with the x-ray beam 230. The monitoring illustratively occurs by the use of radiation pulses 220 which are delivered by Proton-Electron Double-Resonance Imaging or PEDRI technique. By imaging the distribution of free radicals 240 generated by the x-ray beam, PEDRI may be used in accordance with this disclosure to determine the path taken by the x-ray beam 230 through the tissue. The PEDRI data provides real-time data useable by a computer (not shown) that is configured to manage the therapy apparatus (i.e., the x-ray apparatus 212) and the monitoring apparatus 214. Advantageously, the disclosed system may use PEDRI data to provide realtime adjustments to the trajectories and geometry of the x-ray beam 230 generated by the therapy apparatus during a therapy treatment in order to minimize unwanted damage while maintaining therapeutic effect.

FIG. 2B shows an adjustment having been made to the trajectory and geometry of the x-ray beam 230 as a result of the PEDRI data generated during the earlier part of the treatment shown in FIG. 2A. The adjustment may be an automated adjustment by the computer. The adjustment may also be semi-automatically performed by the computer with the interaction of the therapist.

Through the imaging techniques provided by the monitoring system 200 of this disclosure, the system is able to visualize directly or with the aid of a therapist the path taken by the x-ray radiation beam 230 within the body, creating short-term and long-term latent images of the beam trajectory, identifying the tumor, and making real-time changes in beam trajectory in relation to the tumor and organs during treatment. The result is an x-ray radiation treatment system that is more strategic, more effective, and causes less damage to healthy tissue during an x-ray radiation treatment.

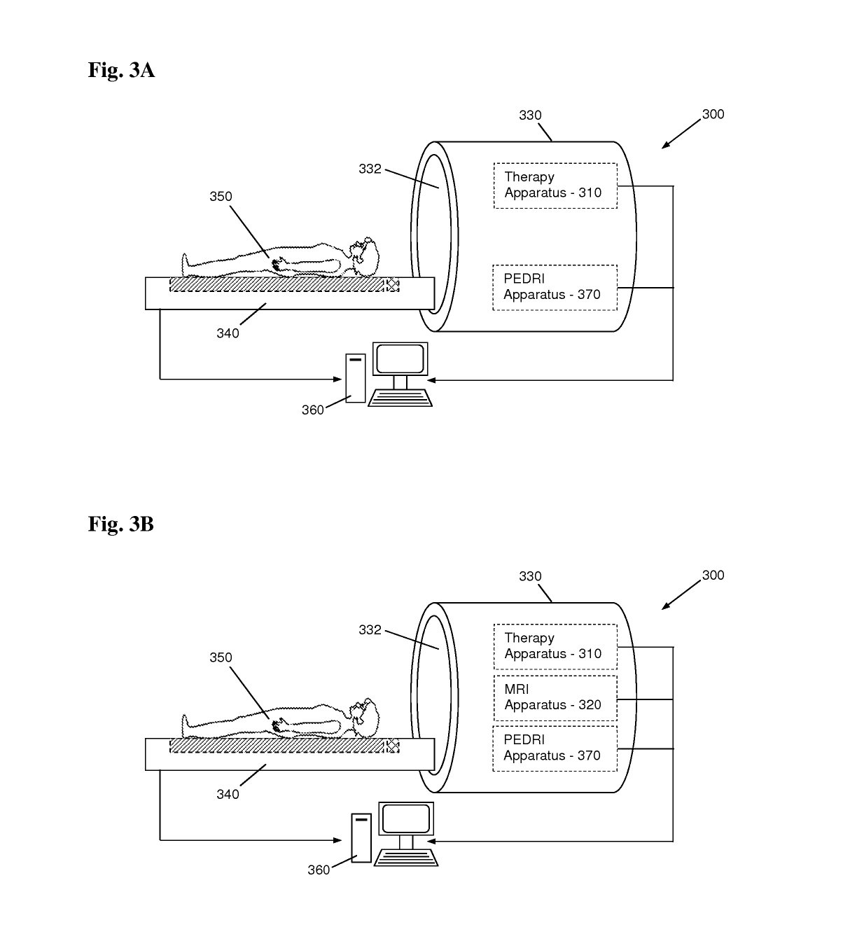

FIG. 3A shows a radiation therapy system 300 according to one illustrative example of this disclosure. The radiation therapy system 300 includes a gantry 330 and a patient couch 340. The gantry 330 includes a therapy apparatus 310 and a PEDRI apparatus 370. The therapy apparatus 310 includes a main magnet, like a bore magnet (not shown) and a radiation source such as x-ray, particle beams, etc. The magnet includes a magnet radiation translucent region (not shown) which allows the radiation beams to pass through the magnet. The radiation source is mounted on a rail system (not shown) that allows the radiation source to be rotated about the translucent regions of the magnet. A collimator (not shown) is used to shape the beam of radiation to localize the treatment to a target area. A controller 360 is used to control the therapy apparatus 310.

As previously described, PEDRI is the imaging version of the. Overhauser experiment In the Overhauser experiment a combination of nuclear magnetic resonance (NMR) and electron spin resonance (ESR) is applied to tissue. The NMR signal is obtained by applying pulses of radiowaves at the NMR frequency. At the same time, the free radical's unpaired electrons are irradiated by applying an irradiation (radiowave or microwave) at the ESR frequency. If there is good magnetic coupling between the unpaired electrons and the water hydrogen nuclei, the ESR irradiation can cause a transfer of polarization from the electrons to the nuclear spins, resulting in an amplification or “enhancement” of the measured NMR signal.

An illustrative PEDRI apparatus of the disclosure illustratively includes an RF coil assembly to receive Proton-Electron Double-Resonance signals from the tissue and hardware to generate and apply a radio frequency for NMR and ESR irradiation.

The hardware to generate the NMR radiation is a high field magnet of generally less than 1.5 or 1 tesla on up. In practical applications, the frequency of the NMR may be similar to VHF and UHF television broadcasts (60-1000 MHz).

The hardware to generate the ESR may be a low field magnet. This is unlike the high field magnet used to generate the NMR or used in magnetic resonance imaging (MRI) systems such as described in FIG. 1. This is because the ESR frequency may be about 659 times the nuclear magnetic resonance (NMR) frequency in a given magnetic field. For example, the low field magnetic of the PEDRI apparatus may be about 0.01 tesla which brings the ESR frequency to about 280 mega-hertz (MHz). A 1 tesla magnet of an NMR or MRI system would bring the ESR frequency to about 28 giga-hertz which may not provide sufficient tissue penetration, and, given sufficient power, may be damaging to the tissue of the patient.

It will be appreciated that the low field magnet used to generate ESR may be a single magnet. Alternatively, any number of individual magnets may be used collectively to attain needed field strength(s) and field uniformity for the ESR. Similarly, the high field magnet used to generate the NMR or used in magnetic resonance imaging (MRI) systems such as described in FIG. 1 may be a single magnet. Alternatively, any number of individual magnets may be used collectively to attain needed field strength(s) and field uniformity for the NMR or magnetic resonance imaging (MRI) systems. In either instance where a collection of individual magnets is used to attain the needed field, it will be appreciated that as the field strength is changed, power to each magnet may illustratively be ramped up or down individually. In the end, the total magnetic field is generally what counts.

The ramp up and down of the weak fields used to create ESR and the strong fields used to create the MRI may introduce some lag between the resulting PEDRI image and overlaid MRI image; with the lag determined by the ramp speed of the slowest magnet.

In an alternative embodiment, fast field cycling may be used to minimize this lag. More particularly, an MRI system has a large, very strong, usually superconducting, magnet that aligns all of the nuclear spins in one direction (additional smaller magnets may also be used to establish field gradients). Then, a separate, much weaker and much faster, coil produces transient perturbations to the field. These small perturbations induce the resonances in the aligned protons which are measured. Therefore, an MRI system has both a slow, big magnet and a small, fast coil. PEDRI, just as MRI, needs a large magnet to align the spins, as well as a coil to create the transient perturbations. Unlike MRI, the coil operates at different frequencies than the MRI coils, and the large magnet in PEDRI is generally weaker than the strong magnet used in MRI. In both cases, the large magnet thus aligns all of the nuclear spins; with the weaker and faster, coil associated with each large magnet producing transient perturbations to the field.

“Field cycling” generally refers to changing the field produced by the large superconducting magnet, not the field of the fast coil. To obtain a lower field in the large magnet, the current running through the large magnet may be reduced (i.e., field cycling). Illustratively, the current through both the large magnet used to generate the MRI and the large magnet used to produce the PEDRI may be field cycled. Alternatively, the current through one large magnet may be field cycled while the current through the other magnet may be left unchanged. A complex arrangement of hardware drivers may be used to create fast cycling and the hardware and the methodologies for doing this field cycling on large magnets are well known in the art. This fast field cycling of the large magnets may be used to minimize some lag that may exist between a PEDRI image and an overlaid MRI image and is an alternative embodiment of this disclosure.

In the illustrative fast field cycling embodiment previously mentioned, current in the weaker coils is ramped up and down very fast. Advantageously, this fast field cycling of current through the coil associated with each large magnet may minimize the lag between the transient perturbations to the field caused be each coil; thereby minimizing some lag that may exist between a PEDRI image and an overlaid MRI image. The fast field cycling of this illustrative embodiment takes up relatively little space, and may thus provide more flexibility in reducing lag between images than may be possible using the fast field cycling of the large magnets also herein disclosed.

Referring still to FIG. 3A, a patient 350 lying down on the top of the patient couch 340 is placed inside an examination region 332 of the gantry 330. The therapy apparatus 310 provides a radiation beam for therapy treatment. The PEDRI apparatus 370 applies selected magnetic field gradient pulses across the imaging region appropriate to a selected magnetic imaging or spectroscopy sequence. The data received from the PEDRI apparatus 370 is processed by controller 360. The PEDRI data controller 360 can perform various functions including free radical imaging reconstruction, Proton-Electron Double-Resonance spectroscopy, etc. Reconstructed PEDRI images, spectroscopy readouts, and other processed PEDRI data may be stored in memory (not shown) of the controller 360 and displayed on a graphical user interface.

The controller 360 may provide control of the therapy apparatus 310, the PEDRI apparatus 370 and provide additional functions. In some embodiments, control functions are distributed over a plurality of controllers.

Prior to the treatment, a plan for the treatment is developed and finalized. The amount of radiation used in radiotherapy is determined depending on tumor type, size, stage, etc. The radiation beam trajectories and geometries are determined from 3-D images. The images may be generated using computed tomography (CT), x-ray, x-ray fluoroscopy, ultrasound, MR, etc. The current target volume positions are aligned to the coordinate system of the radiation source assembly. Surgical implanted markers and/or other indicators may also be used for alignment. During treatment, the actual PEDRI data of the target and non-target volumes is monitored and may be periodically, aperiodically, or continuously compared to PEDRI data taken earlier in the current treatment. Any misalignment between the PEDRI data of the target and non-target volumes taken at two or more points of time may provide data for use by the controller in correcting the trajectory and shape of the x-ray radiation beam to minimize damage to healthy tissue while maintaining treatment.

FIG. 3B shows a radiation therapy system 300 of FIG. 3A, but with a few modifications according to another illustrative example of this disclosure. The elements in the radiation therapy system 300 of FIG. 3B are the same number as the elements in FIG. 3A. In addition, the radiation therapy system 300 of FIG. 3B includes an MR apparatus 320.

In FIG. 3B, the PEDRI apparatus 370 includes a low field magnet hardware to generate the ESR but illustratively contains no high field magnet to generate the NMR. Instead, the high field magnet to generate the NMR is provided by the MRI apparatus 320, which has like function and operation as the MRI apparatus 120 in FIG. 1. Alternatively, the PEDRI apparatus 370 may include the high field magnet as in the PEDRI apparatus 370 of FIG. 3. In either case, the controller 360 of the MRI apparatus 320 provides the PEDRI apparatus 370 of this disclosure with various additional functions. These include MR image reconstruction, magnetic resonance spectroscopy, reconstructed magnetic resonance images, spectroscopy readouts, and other processes. In addition, after treatment, the actual dose delivered to each voxel of the target and non-target volumes determined based on pre-treatment and post-treatment MRI images may be correlated with the PEDRI data to provide additional data for the treatment plan.

The monitoring apparatus 310 in FIGS. 3A, 3B respectively have been described as a noninvasive form of a functional magnetic resonance imaging (fMRI) device known as proton-electron double-resonance imaging (PEDRI) for monitoring free radicals generated in the tissue of the patient. It will be appreciated from this disclosure that the monitoring apparatus may be any fMRL apparatus configured for monitoring of free radicals generated in the tissue of the patient. In other illustrative examples, the monitoring apparatus is an apparatus configured for monitoring of biomarkers that indicate a trajectory of the therapeutic beam as described in greater detail below.

In each of FIGS. 2 and 3, the PEDRI apparatus monitors and captures the trail of the free radicals caused by the x-ray radiation. This allows the PEDRI imaging to visualize for either or both the therapy apparatus and a therapist the path taken by the x-ray radiation beam through the patient. Also advantageously, the PEDRI capture of this trail through slices of time allows the creation of a temporal evolution of these radiation-induced free-radical species monitored by PEDRI. This allows both the therapy apparatus and the therapist to see how the path of the beam changes over time. In other words, the PEDRI data generated by the system of this disclosure provides a time-resolved record of each beam trajectory for use in changing the trajectory and/or shape of the x-ray beam, the dosage levels delivered by the beam to the target and non-target tissue, the treatment plan, etc. Further, identification of the free radicals themselves may provide a temporal aspect to the PEDRI data, since the free radicals may change form in time. For example, the free radicals may change from highly reactive ones to slowly evolving ones. Thus, the type of free radicals identified by PEDRI in a particular volume may also provide temporal information. In other words, the type of free radicals identified in a time slice of PEDRI data may be indicative of the time of irradiation since the type of free radicals may be indicative of how long ago the irradiating beam has passed through a respective volume of tissue. The temporal associations that are enabled by assessment of the type of free radicals in the PEDRI data may be used alone or together with the tracking of time-slices of PEDRI data previously described to provide time-resolved PEDRI data.

In each of FIGS. 2 and 3, the controlling computer may be configured with appropriate apparatus including communications equipment, equipment interfaces (such as an interface with the therapy apparatus and another interface with the monitoring apparatus), a user interface (including, for example, a keyboard, a graphic display, a pointing device and the like). The controller may further be configured with software (that is, for example, machine readable instructions stored on machine readable media, the instructions for controlling the radiation therapy system as well as performing other intended functions such as interfacing with users and providing output).

By engaging the disclosed monitoring apparatus during operation of the therapy apparatus, the radiation therapy system of this disclosure provides accurate and precise control over radiation therapy. More specifically, ongoing radiation therapy provided by the therapy apparatus may be monitored with the monitoring apparatus and adjusted on a continuing basis by, for example, the controller. Adjustment may be performed by comparing an assessment of the ongoing radiation therapy to a treatment plan designed for the patient.

It should be noted that the exemplary embodiments make use of x-rays and monitor coincident generation of free radicals. However, this is merely illustrative and is not limiting. For example, other radiation sources as well as other biomarkers may be used. For example, gamma radiation may be used for therapy. Biomarkers may include indications produced by for example, use of contrast agents. Aspects of some non-limiting and additional embodiments are discussed further herein.

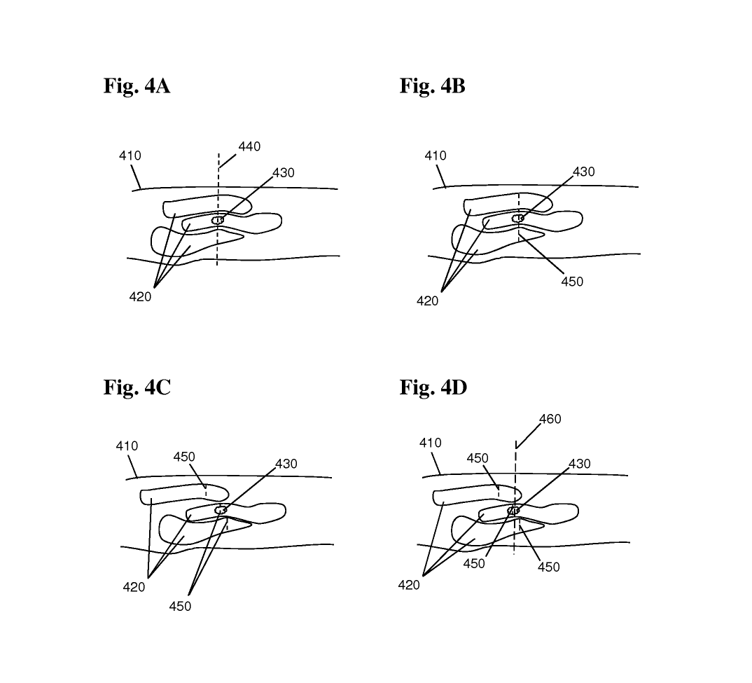

FIGS. 4A-4D, collectively referred to herein as FIG. 4 illustrate how the system of this disclosure may account for movement of tissue in the patient and provide for correction of the trajectory and shape of a radiation beam being delivered In this illustration, movement of organs relative to a trajectory of a therapeutic beam is depicted. In FIG. 4A, a cross-section of the patient 410 is shown. Included within the patient 410 is a plurality of organs 420. Seated within one of the organs 420 is a target 430, such as a tumor. In this illustration, a first pulse 440 of a therapeutic beam traverses the plurality of internal organs 420 and intersects with (i.e., irradiates) the target 430. Generally, the target 430 may be characterized as a particular volume of interest within the patient 410. Although the target 430 is generally referred to herein as a tumor, the target 430 may include any type of tissue where treatment with radiation therapy is desired. In FIG. 4B, monitoring of the patient 410 provides for depiction of biomarkers 450, such as free radicals generated by the first pulse 440 of radiation as described above, through the organs 420. More specifically, the biomarkers 450 depict a trajectory of the first pulse 440 of the therapeutic beam that was applied to the patient 410. As shown in FIG. 4C, movement or adjustment of the patient 410 may result in translocation of the internal organs 420. Accordingly, the biomarkers 450 that appear in a first organ 420 have shifted relative to biomarkers 450 that appear in a second organ 420. By application of monitoring results, such as the PEDRI data generated according to this disclosure, and as shown in FIG. 4D, orientation of a second pulse 460 of the therapeutic beam may be adjusted such that the second pulse 460 also intersects with and irradiates the target 430, despite the target 430 having shifted from its original location.

In practice, PEDRI may be carried out using standard MRI hardware and software, such as described in FIG. 3B, with the addition of hardware to irradiate the free-radical-of-interest's EPR resonance. But as described in FIG. 3A, MRI hardware is not required. A high field magnet may be used for the hardware to generate the NMR radiation. For example, and as previously explained, the hardware to generate the ESR radiation and the NMR radiation may be a single magnet or set of magnets whose field can be cycled between weak and strong fields to provide the ESR and NMR images, respectively. Fast field cycling may be used to minimize some lag that may exist between a PEDRI image and an overlaid MRI image.

The intensity of the PEDRI signal (i.e., the brightness of the image) and the color, correlates to the amount and/or the type of free radicals present, and thus signal intensity and color may be used to quantify radiation dose to the tissue at each imaged volume element. The images of radiation beam trajectories can be readily over-laid onto images of the target 430 (e.g., the tumor) and surrounding tissue by simultaneously using different images acquired using other imaging modalities. For example, the PEDRI images may be over-laid over images generated using computed tomography (CT), x-ray, x-ray fluoroscopy, ultrasound, MR, etc. to provide additional information for use in minimizing harm to tissue while maintaining therapy treatment.

In some embodiments, a secondary beam may be combined with the x-ray or other primary radiation beam such that both beams have the same trajectory in the patient. The secondary beam may be used for pre-alignment of the primary beam. For example, the secondary beam may provide for stimulating an observable response along a common trajectory before any radiation is applied. This embodiment has the advantage that the secondary beam may be chosen to cause little damage to the tissue, and may thus be kept active throughout alignment, while providing real-time imaging of the beam trajectory.

Secondary beams may be chosen from any region of the electromagnetic spectrum deemed appropriate. For example, secondary beams may include radiation that can penetrate tissue without causing substantial damage. Examples of such secondary collimated beams include low-level X-rays and microwave signals.

In some embodiments, a secondary beam that includes frequencies of microwave radiation is particularly useful, because the excitation frequencies for Electron Spin Resonance (ESR) and PEDRI measurements fall in the microwave spectrum. A secondary beam of the proper microwave frequency can stimulate resonance in electrons, thus enabling direct visualization of the secondary beam by PEDRI, permitting pre-alignment of the beam to the tumor using PEDRI data taken on the secondary beam before any radiation is applied. In some of these embodiments, Microwave Amplification by Stimulated Emission of Radiation (MASER) may be used to produce collimated, single-frequency, narrow beams in the microwave region of the electromagnetic spectrum.

Aspects of some additional embodiments are now presented.

In order to increase the latency of the image over direct visualization of free radicals, monitoring of the trajectory of a radiation beam may be accomplished by monitoring of biomarkers that are created by free radicals. First, it should be recognized that the body produces numerous compounds that upon exposure to free radicals may be detected through imaging. One technique for imaging such compounds includes use of magnetic resonance imaging (MRI) configured to monitor free radical triggered biomarkers, and may include incorporation of at least one contrast agent. Contrast agents may be selected according to a specific affinity for selected compounds (i.e., biomarkers). Some non-limiting examples of compounds that may be used as biomarkers are provided herein. Table 1 compares some relevant classes of biomarkers with analysis requirements.

TABLE 1
Comparison of Biomarkers for Free Radical Damage

| | | | | | Oxi- |
|---|---|---|---|---|---|
| | | | | | dized |
| | | | Iso- | 8-OH- | amino |
| Requirement | Alkanes | Aldehydes | prostanes | dG | acids |
| Speed of analysis | − | ± | − | − | − |
| Sensitivity | ± | ± | ± | ± | ± |
| Ease of analysis | − | ± | − | − | ± |
| Non-invasiveness | + | + | − | ± | − |
| Confounding factors | − | − | ± | ± | ± |
| Interfering | − | − | ± | ± | ± |
| background | | | | | |
| Specificity for effect | ± | ± | + | ± | ± |
| Damage correlation | + | + | + | ± | ± |
| Application in | + | ± | − | ± | ± |
| humans | | | | | |

In some embodiments, particular regard may be given to physiological dynamics that occurred during radiation therapy. For example, some embodiments may provide for monitoring through evaluation of bursting of vesicles.

A vesicle is a lipid bilayer membrane that encloses a volume. In general, vesicles are prone to damage from radiation exposure, and will burst when adequately exposed to radiation. Accordingly, some embodiments of monitoring take advantage of this effect. That is, some embodiments of monitoring call for encapsulation of particles of a contrast agent (e.g., iron oxide nanoparticles) inside a plurality of vesicles. A multitude of contrast agent containing vesicles may be placed in the blood stream. Upon exposure to radiation, the vesicle bursts, releasing the contrast agent. These nanoparticles then bind to surrounding tissue, and may be imaged through MRI or other techniques.

In some embodiments, the vesicle may burst solely due to exposure to radiation. In some other embodiments, the bilayer membrane may contain a radiation-sensitive component that destabilizes the bilayer membrane upon exposure to lower-level radiation. Some mechanisms that serve to destabilize the bilayer membrane include release of free-radicals, generation of heat, or nano-bubble nucleation. In some further embodiments, instead of bursting, the bilayer membrane may become permeable to the contrast agent upon exposure to radiation.

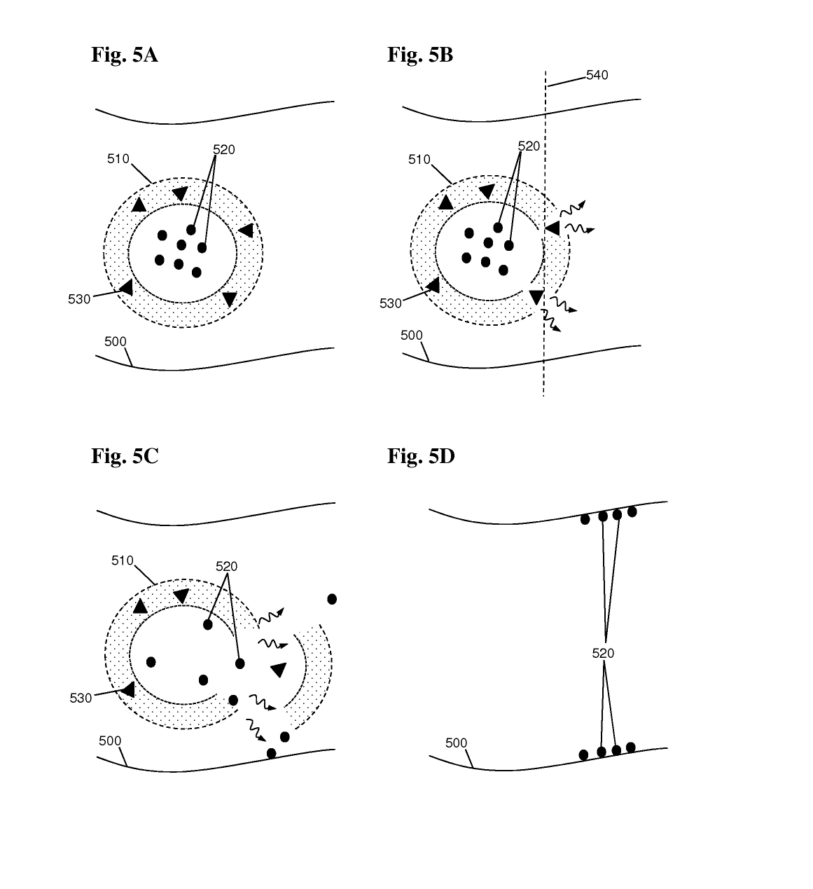

FIGS. 5A-5D, collectively referred to herein as FIG. 5, illustrates process steps involved in use of vesicles for distributing contrast agent. In FIG. 5A, a vesicle 510 is depicted. In this example, the vesicle 510 encloses a multitude of nanoparticles that include contrast agent 520. The vesicle 510 is solubilized into tissue such as the bloodstream, such that it has a low affinity to walls 500 of the bloodstream. In some embodiments, a bilayer membrane of the vesicle 510 further includes a radiation sensitive agent 530. In FIG. 5B, the vesicle 510 is exposed to a beam of radiation 540. Upon exposure to the radiation 540, the bilayer membrane of the vesicle 510 bursts. Disruption of the membrane occurs either directly as a result of exposure to the radiation 540, or through operation of the radiation sensitive agent 530. Upon bursting, as shown in FIG. 5C, the vesicle 510 releases the contrast agent 520 into the surrounding volume (i.e., into the bloodstream). As shown in FIG. 5D, once released, the nanoparticles of contrast agent 520 become attached to the surrounding tissue (such as the walls 500) through at least one of specific bonding and nonspecific bonding.

Another technique for in-vivo monitoring of radiation beams includes relying upon dissociation of an adduct to provide the contrast agent. In general, an “adduct” is a non-covalently bonded aggregate of two (or more) chemical species. In some of these embodiments, one of the “parts” of the adduct acts as a solubilizing agent, making the adduct soluble inside a respective tissue of the patient, for example, in the patient's bloodstream or gastrointestinal tract. The second part of the adduct is a contrast agent that is not, by itself, soluble in the tissue (e.g., the bloodstream or GI tract), but which may be imaged by one of several medical imaging methods such as MRI or ultrasound. When the adduct is exposed to radiation, or to a by-product of radiation exposure, such as free-radicals, the adduct breaks apart. The contrast agent then precipitates out of solution and becomes attached to the tissue. In this way, the contrast builds up in areas near radiation exposure.

Some classes of adducts that may be useful include surfactant-dye aggregates and nanoparticles. Of the latter, ferric-ceria compounds may be useful, as they are sensitive to free radical exposure, and in some cases are approved for use in-vivo. Another example of a potentially useful adduct is a metallic nanoparticle (e.g., iron, a strong MRI contrast agent), which has a radiation-sensitized coating that solubilizes the nanoparticle in-vivo, but degrades upon exposure to radiation causing the iron to fall out of solution.

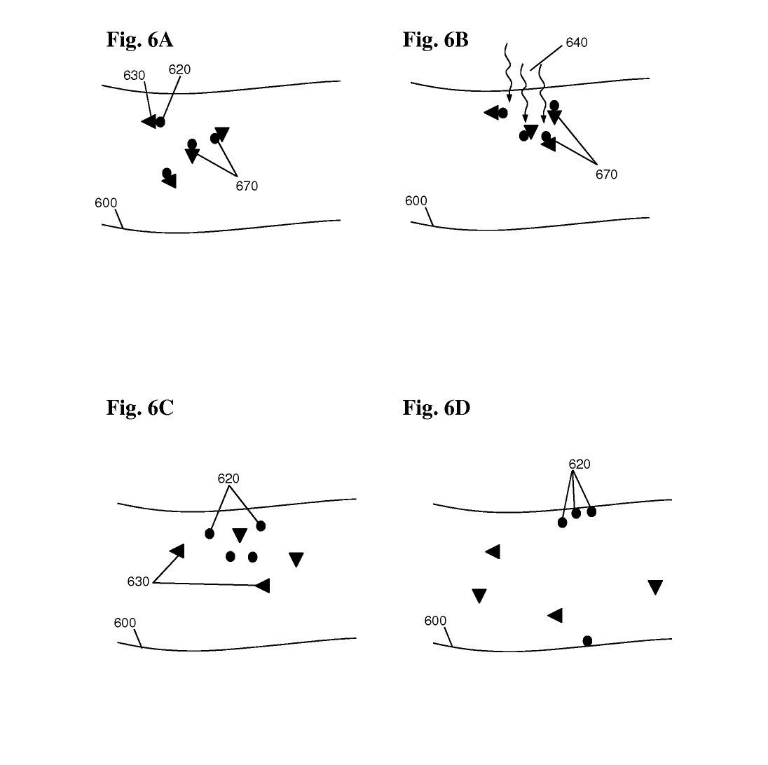

FIGS. 6A-6D, collectively referred to herein as FIG. 6, illustrates process steps involved in dissociation of an adduct for distributing contrast agent. As shown in FIG. 6A, the process begins with introduction of adducts 670 into a tissue or fluid such as the bloodstream. Each adduct 670 is a combination of the contrast agent 620 and a solubilizing part 630. Upon exposure to the beam of radiation 640 (FIG. 6B), the adduct 670 will break apart (FIG. 6C). When the adduct 670 breaks apart, the contrast agent 620 is separated from the solubilizing part 630 of the adduct 670. As shown in FIG. 6D, once the contrast agent 620 is made available, the contrast agent 620 becomes attached to the surrounding tissue (such as the walls 600).

Yet another technique for monitoring radiation dose includes monitoring generation of nano-bubbles. As used herein, the term “nano-bubbles” generally refers to gas bubbles with diameters as small as about 100 nm. Nano-bubbles may be generated by the radiation beam, and visualized by ultrasound, which is a simple and comparatively less expensive technique for visualization of radiation dose.

In some embodiments, nano-bubbles may be generated around metal nanoparticles in-vivo by creating a plasmon (oscillating electrons on the surface of a metal nanoparticle). This may be accomplished with pulsed laser light. A wavelength that is selected for the laser is generally matched to that of the plasmon. When the wavelength strikes the plasmon, heat is generated and results in a bubble of air and water vapor. For in-vivo measurements, nanoparticles may be introduced either into the bloodstream, or into the gastro-intestinal tract prior to therapy session.

In one embodiment, a laser of the appropriate wavelength may be collimated with the radiation beam, and used to stimulate surface plasmons along the trajectory of the beams. In another embodiment, the plasmons are generated by the radiation beam itself.

In a further technique for monitoring radiation dose, chemiluminescent probes used to provide non-invasive in-vivo imaging of free radical production was demonstrated using chemiluminescent probes in combination with an imaging system configured for monitoring bioluminescence.

In yet another technique for monitoring radiation dose, imaging of damage to proteins is performed. Besides creating free radicals, radiation also causes damage to proteins and nucleic acids. By using specific-binding contrast agents and imaging techniques as are known in the art, damage to these proteins and nucleic acids may be visualized. That is, in some embodiments, contrast agents may be bonded to damaged DNA or RNA and activated upon exposure to free radicals. Advantageously, by focusing on nucleic acids or proteins as indicators (biomarkers) of radiation exposure, a great number of contrast agents that are commercially available may be used. Further, the low mobility of the nucleic acids and most proteins, in some instances will yield longer image persistence times of imageable features over free-radical tracking methods described above.

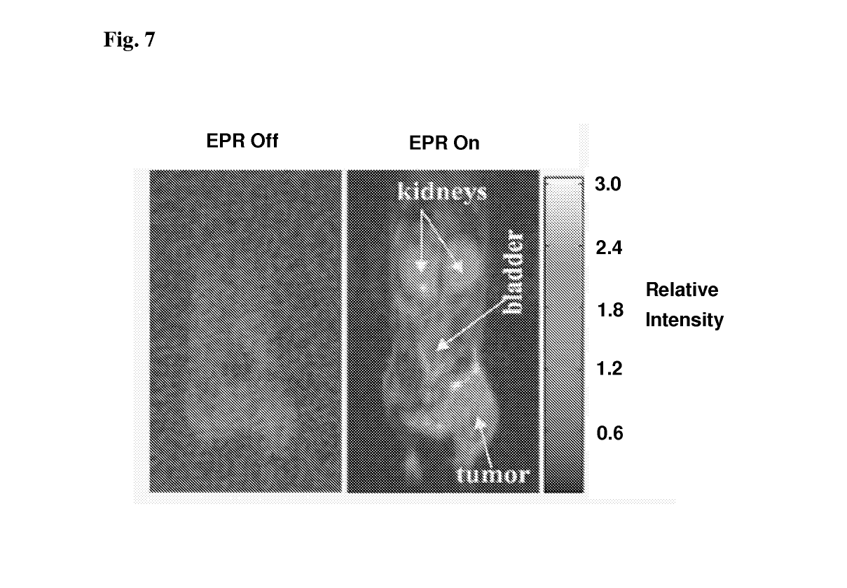

Turning to FIG. 7, it may be seen that the techniques disclosed herein may further be used with traditional imaging of target tissue techniques. In FIG. 3B, the use of this disclosure with an MRI apparatus was described. FIG. 7 shows the oxygenation levels of tissue generated by the MRI apparatus used with the system of this disclosure with the EPR excitation feature of the PEDRI turned off and on. In this illustration, enhanced imaging (right image) made possible using the disclosed system with PEDRI apparatus turned on shows substantially more information than a traditional MRI image (left image). Thus, the disclosed system allows a tumor to be more readily identifiable according to higher levels of oxygenation.

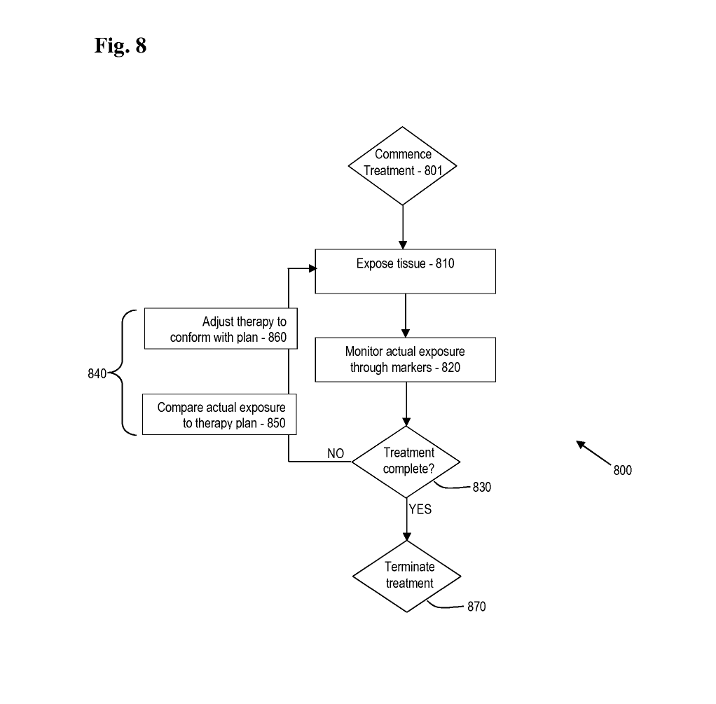

Turning to FIG. 8, an exemplary method 800 for performing radiation therapy is shown. The exemplary method 800 for performing radiation therapy begins by commencing treatment 801. Commencing treatment 801 generally involves patient set up procedures that are performed for other forms of radiation therapy. For example, commencing treatment 801 involves verifying patient identity, positioning the patient with regards to therapeutic equipment and adjusting settings for the therapeutic equipment area. Once the initial setup has been completed, exposing tissue 810 begins. Exposing tissue 810 may involve, for example, energizing an x-ray system, exposing the radiation source, or the like. Generally, exposing tissue 810 simply calls for delivering a therapeutic beam of radiation to the tissue of the patient. Once exposing tissue 810 is underway, monitoring biomarkers 820 commences. Generally, monitoring biomarkers 820 is performed in real time, substantially real-time or nearly real-time. That is, monitoring biomarkers 820 generally proceeds at a pace that is adequate for adjusting the exposing tissue 810 in a way that is meaningful to enhance the efficacy of the therapy.

The method for performing radiation therapy 800 proceeds until such time as a prescribed radiation dose has been delivered to the tissue. Accordingly, testing for completion 830 is also performed in a real-time, substantially real-time or nearly real-time basis. Generally, testing for completion 830 calls for comparing an estimated dose delivered to the prescribed dose. The delivered radiation dose may be estimated according to monitoring results, calculations based on machine output, and by other similar information.

If testing for completion 830 determines that therapy has not been completed, then adjusting therapy 840 may be performed. Generally, adjusting therapy 840 calls for comparison 850 of delivered radiation dose (as ascertained by monitoring biomarkers 820) to a therapy plan. Results of the comparison may be used for adjusting therapy 860 according to at least one of delivery of the therapeutic beam, positioning of the therapeutic beam, and by other similar factors. In some embodiments, adjusting therapy 860 may involve adjusting pulse length of delivery pulses, energy of the therapeutic beam of radiation, and other similar characteristics.

Generally, the method for performing radiation therapy 800 proceeds until testing for completion 830 has determined that the prescribed radiation dose has been delivered to the tissue, at which point, termination of treatment 870 will be initiated by a clinician, automatically by the therapy system, or in a similar manner.

The method for performing radiation therapy 800 may include additional steps and features. For example, the method for performing radiation therapy 800 may include output of progress information to a user or another system, and may further receive input or control from an external source (such as the user or another system).

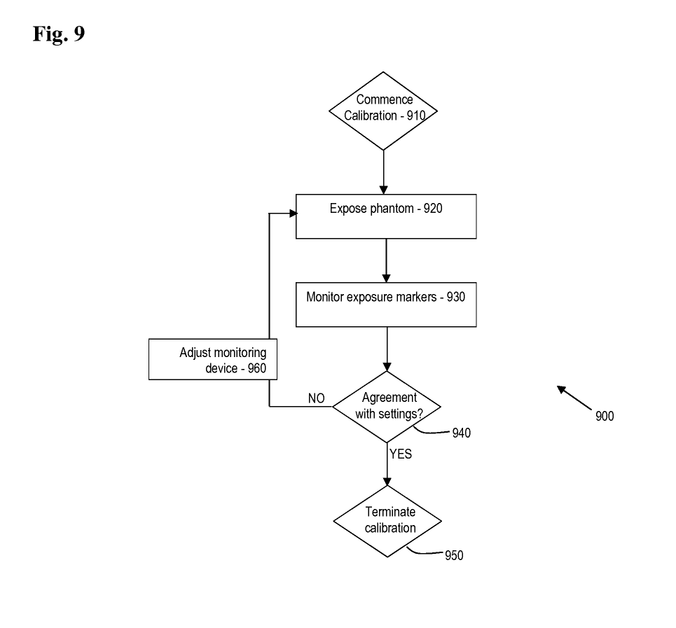

Referring now to FIG. 9, there is shown an exemplary method 900 for calibration. Generally, the radiation therapy system 900 is calibrated after calibration or characterization of the therapy apparatus (for example, the x-ray apparatus that will deliver the therapeutic beam of radiation to the tissue of a patient).

The method for calibration 900 starts with commencing calibration 910. Commencing calibration 910 may include verification of system setup, placement of calibration equipment, such as a tissue equivalent phantom and the like. Generally, exposing the phantom 920 results in generation of exposure markers, for example, free radicals. Similar to the method for providing radiation therapy 800, monitoring of exposure markers 930 is performed. In a following step, testing for agreement 940 involves comparing results of the monitoring of exposure markers 930 with a known quantity of radiation delivered to the phantom. The known quantity of radiation may be ascertained by traditional dose calculations, while relying on a fresh calibration or characterization of the therapy equipment. If testing for agreement 940 determines that results provided by the monitoring apparatus are not in agreement with dose calculations, then a process of adjusting the monitoring device 960 may be performed. Generally, the method for calibration 900 continues until such time as testing for agreement 940 determines there is adequate correlation between radiation dose delivered by the therapy apparatus and radiation dose estimated by the monitoring apparatus.

In view of this disclosure, it will be seen that technologies are generally described for visualization of beam trajectories during radiation therapy in order to determine the path taken by the radiation beam of radiation through the tissue.

This disclosure describes several systems and methods for imaging a trajectory of radiation beams and measuring local radiation dose in each imaged volume unit (or “voxel”, the three-dimensional (3D) analogs to “pixel”) in order to determine the path taken by the radiation beam of radiation through the tissue.

An exemplary embodiment includes use of PEDRI (Proton-Electron Double-Resonance Imaging), which is a sensitive imaging method. PEDRI is capable of detecting and quantifying local radiation exposure in-vivo by visualizing free radicals created in the body by radiation exposure.

By monitoring temporal evolution of these radiation-induced free-radical species, techniques disclosed herein may provide a time-resolved record of the path taken by each beam trajectory.

When combined with other modes of imaging, using the same or different imaging hardware, the techniques may be used to provide a multi-mode imaging system that provides a complete and quantitative picture of the progress of radiation therapy, including high-resolution imaging of the radiation beam trajectories, a quantitative measure of the local radiation dose at each volume element, identification and visualization of a given tumor, all of which may be over-laid onto three-dimensional (3D) images of surrounding tissues and organs.

According to this disclosure, a radiation therapy system includes an x-ray apparatus configured for delivery of a therapeutic beam of radiation to tissue of a patient; and a monitoring apparatus configured for monitoring production of free radicals within the tissue during the delivery. The monitoring apparatus may be a magnetic resonance imaging (MRI) system. The magnetic resonance imaging system (MRI) may be configured for proton-electron double-resonance imaging (PEDRI). The magnetic resonance imaging system may be further configured to monitor oxygenation levels in the tissue.

The system may further include an apparatus for delivery of a secondary beam of radiation. The tissue receiving the therapeutic beam of radiation may be at least one of an organ, a tumor, and tissue surrounding a tumor. The monitoring apparatus may be further configurable for monitoring an indication from a contrast agent. The monitoring apparatus may be further configurable for monitoring movement of internal organs. The monitoring apparatus may be further configurable for monitoring a trajectory of the therapeutic beam.

A management system may be provided to the system for controlling the delivery according to monitoring information. The management system may be configured for controlling the delivery in real time. The management system may include a computing system. The computing system may include a computing program product stored on machine readable media, the product configured for receiving monitoring information and controlling the delivery.

The x-ray apparatus of the system may include components for adjusting a positioning of the therapeutic beam during the delivery. A user interface may also be provided to the system. The user interface may provide at least one of: a visual representation of the beam in relation to the tissue; an image that varies in at least one of brightness and color according to a quantity of free radicals present in the tissue; at least one image as an overlay to an image of the beam; and, an image of the tissue.

The monitoring apparatus of the system may include a contrast agent suited for disposition in the tissue and an imaging device for imaging the tissue. The contrast agent may include at least one of a dispersion of nanoparticles and a surfactant-dye aggregate. The imaging device may include at least one of an ultrasound device and a magnetic resonance imaging device.

A computer program product for controlling radiation therapy may include machine executable instructions stored on machine readable media. The product may include instructions for receiving information descriptive of production of free radicals within tissue of a patient during radiation therapy, the free radicals resulting from a therapeutic beam of radiation. The production may be compared to a treatment plan. The radiation therapy may be controlled according to results of the comparison. The instructions may include instructions for recording imaging data. The instructions may include instructions for collecting the descriptive information. The instructions may include instructions for reporting results from the comparing. The instructions may include instructions for communicating with a user interface. The instructions may include instructions for performing the receiving, comparing and controlling in real-time.

A method for calibrating a radiation therapy system may include selecting a radiation therapy system that includes an x-ray apparatus configured for delivery of a therapeutic beam of radiation to tissue of a patient and a monitoring apparatus configured to monitor production of free radicals within the tissue during the delivery. The delivery of the therapeutic beam may be adjusted to correlate with a standard. The standardized therapeutic beam may be delivered to a tissue equivalent phantom. The production of the free radicals may be monitored within the phantom. The monitoring results may be adjusted to correlate to the standardized therapeutic beam.

A method for providing radiation therapy may include obtaining a treatment plan for delivering radiation to a tissue of a patient. An x-ray therapy system is selected that includes an x-ray apparatus configured for delivery of a therapeutic beam of radiation to tissue of a patient and a monitoring apparatus is configured for monitoring production of free radicals within the tissue during the delivery. The therapeutic beam of radiation is delivered to the patient with the radiation therapy system according to the treatment plan. The monitoring may be of the production of free radicals. The monitoring may include quantifying the free radicals. The quantifying may include determining a signal intensity of a proton-electron double-resonance imaging (PEDRI) signal. The monitoring may further include monitoring of a secondary beam of radiation. The monitoring may further include observing oxygenation levels within the tissue.

A graphic output of the therapeutic beam may be provided. The movement of an organ within the tissue may be monitored. A graphic output of the movement may be provided. A contrast agent may be introduced into the tissue. The delivery may be adjusted according to a comparison of monitoring information and the treatment plan. At least one of monitoring the production of free radicals and adjusting the delivering in substantially realtime may also be done.

In other illustrative embodiments, a radiation therapy system includes a therapeutic apparatus configured for delivery of a therapeutic beam of radiation to tissue of a patient and a monitoring apparatus configured to monitor production of biomarkers within the tissue during the delivery. The biomarkers may include at least one of free radicals, burst vesicles, dissociation of an adduct and an indication of changes in and/or damage to proteins, cells, lipids, nucleic acids, and other tissues or biomolecules. A management system for controlling the delivery according to monitoring information may be provided. The management system may be configured for controlling the delivery in real time. The management system may include a computing system. The computing system may include a computing program product stored on machine readable media, the product configured for receiving monitoring information and controlling the delivery. The therapeutic apparatus may include at least one device for providing radiation in a predetermined range of the electromagnetic spectrum. The predetermined range may include gamma ray and x-radiation. The monitoring apparatus may be a magnetic resonance imaging system (MRI) configured for proton-electron double-resonance imaging (PEDRI). The magnetic resonance imaging system may be further configured to monitor oxygenation levels in the tissue. The tissue may include at least one of an organ, a tumor, and tissue surrounding a tumor. The system may include a user interface. The user interface may provide at least one of: a visual representation of the beam in relation to the tissue; an image that varies in at least one of brightness, color and/or numerical value according to a quantity of free radicals present in the tissue; at least one image as an overlay to an image of the beam; and, an image of the tissue.

The monitoring apparatus may include a contrast agent suited for disposition in the tissue and an imaging device for imaging the tissue, and wherein the contrast agent includes at least one of a dispersion of nanoparticles and a surfactant-dye aggregate. The imaging device may include at least one of an ultrasound device and a magnetic resonance imaging device. The monitoring apparatus may include a bioluminescence imaging system and a plurality of chemiluminescent probes.

A method for calibrating a radiation therapy system includes selecting a radiation therapy system that includes a therapeutic apparatus configured for delivery of a therapeutic beam of radiation to tissue of a patient and a monitoring apparatus configured to monitor production of biomarkers within the tissue during the delivery. The biomarkers may include at least one of free radicals, burst vesicles, dissociation of an adduct and an indication of biomolecule or tissue damage. The delivery of the therapeutic beam is be adjusted to correlate with a standard. The adjusted therapeutic beam is delivered to a tissue equivalent phantom. The production of the biomarkers is monitored within the tissue equivalent phantom. The monitoring results are adjusted to correlate to the adjusted therapeutic beam.

A method for providing radiation therapy includes determining a treatment plan for delivering radiation to a tissue of a patient. A radiation therapy system is selected that includes a therapeutic apparatus configured for delivery of a therapeutic beam of radiation to tissue of a patient and a monitoring apparatus configured to monitor production of biomarkers within the tissue during the delivery. The biomarkers may include at least one of free radicals, burst vesicles, dissociation of an adduct and an indication of biomolecule or tissue damage. The therapeutic beam of radiation is delivered to the patient with the radiation therapy system.

According to this disclosure, PEDRI imaging may be used in conjunction with radiation therapy to provide a variety of beneficial outcomes. For example, PEDRI imaging may provide for: directly visualizing a trajectory of the radiation beam through all organs; quantifying radiation dose for each unit of volume (i.e., tissue) that has been imaged, based on the level of free radicals present (i.e., by evaluating brightness of the image components); as well as detection of radiation beam trajectories for prior and/or ongoing radiation evolutions by monitoring the time evolution of free-radical species from unstable to stable forms.

Additionally, PEDRI imaging may provide for: forming temporal relationships for a radiation beam trajectory based on evaluation of free radical species; pre-alignment of therapeutic devices by visualization of the secondary beam, such as a microwave beam; observation of oxygenation levels of the tissues); and, providing improved insight into progress of radiation therapy through an enhanced graphic image (such as by simultaneous display of beam trajectory information, target information and organ placement).

This disclosure describes several systems and methods for imaging a trajectory of radiation beams and measuring local radiation dose in each imaged volume unit (or “voxel”, the three-dimensional (3D) analogue to “pixel”) in order to determine the path taken by the radiation beam of radiation through the tissue. An exemplary embodiment includes use of PEDRI (Proton-Electron Double-Resonance Imaging), which is a sensitive imaging method. PEDRI is capable of detecting and quantifying local radiation exposure in-vivo by visualizing free radicals created in the body by radiation exposure. By monitoring temporal evolution of these radiation-induced free-radical species, techniques disclosed herein may provide a time-resolved record of the path taken by each beam trajectory. Furthermore, when combined with other modes of imaging, using the same or different imaging hardware, the techniques may be used to provide a multi-mode imaging system that provides a complete and quantitative picture of the progress of radiation therapy, including high-resolution imaging of the radiation beam trajectories, a quantitative measure of the local radiation dose at each volume element, identification and visualization of a given tumor, all of which may be over-laid onto three-dimensional (3D) images of surrounding tissues and organs.

The disclosure results in, among other things: accurate targeting of the radiation beam; accurate monitoring of the location and dosage of all irradiated tissue; observation of the movement of internal organs during therapy; monitoring of the progress of radiation therapy in real time and high resolution; as well as adjusting the trajectory, shape, etc. of a radiation beam of therapy in real-time. Advantageously, techniques disclosed herein are compatible with many existing technologies, including current MRI imaging techniques.

Further, it will be appreciated by one skilled in the art that other techniques for visualizing or imaging radiation dose delivered to the tissue of a patient may be used in conjunction with or in place of PEDRI imaging in order to determine the path taken by the beam of radiation through the tissue.

The PEDRI imaging of this disclosure can be used in conjunction with radiation therapy to: 1. Visualize directly the trajectory of the radiation beam through all organs; 2. Quantify the local dosage of radiation to each imaged unit of volume, based on the level of free radicals present (i.e., brightness of the trajectory image.); 3. Observe latent images of past radiation beam trajectories by monitoring the time evolution of free-radical species from unstable to stable forms; 4. Provide unique “time-stamps” to the latent trajectory images based on the time evolution of free radical species. 5. Visualize a secondary microwave beam in real-time, which can be used for pre-alignment 6. Identify and image the tumor by observing oxygenation levels of the tissues 7. Readily overlay images of beam trajectories and tumor with traditional MRI or other imaging techniques to provide a complete picture of the progress of the treatment in real-time and at high resolution.

As opposed to current methods that align the patient to the radiation beam using stationary (typically external) markers that are distant from the target site, the disclosure visualizes the radiation beam trajectory itself, enabling: 1. Accurate targeting of the radiation beam; 2. Monitoring the location and dosage of all irradiated tissue; 3. Observation of the movement of internal organs; 4. Complete picture of the progress of radiation therapy in real time and high resolution; 5. Compatibility with current MRI imaging techniques.

It will be appreciated that the contrasting agents used in this disclosure are not limiting. One skilled in the art will appreciate that other contrasting agents may be used with this disclosure, including nanobubbles.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-55. (canceled)

56. A radiation therapy system comprising:
a therapeutic apparatus configured for delivery of a therapeutic beam of radiation to tissue of a patient;
a monitoring apparatus configured to monitor production of biomarkers within the tissue during the delivery of the therapeutic beam, the biomarkers comprising at least one of free radicals, burst vesicles, dissociation of an adduct and an indication of changes in or damage to a biomolecule or tissue; and
an apparatus for delivery of a secondary beam of microwave radiation for pre-alignment of the therapeutic beam.

57. The system as in claim 56, wherein the therapeutic apparatus provides radiation in a predetermined range of the electromagnetic spectrum wherein the predetermined range comprises gamma ray, and X-radiation.

58. The system as in claim 56, wherein the monitoring apparatus is a magnetic resonance imaging system (MRI) configured for proton-electron double-resonance imaging (PEDRI).

59. The system as in claim 58, wherein the magnetic resonance imaging system is further configured to monitor oxygenation levels in the tissue.

60. The system as in claim 56, wherein the monitoring apparatus is further configurable for monitoring at least one of movement of internal organs and a trajectory of the therapeutic beam.

61. The system as in claim 56, wherein the monitoring apparatus is further configurable for monitoring an indication from a contrast agent, and wherein the contrast agent comprises at least one of a dispersion of nanoparticles and a surfactant-dye aggregate.

62. The system as in claim 61, wherein the imaging device comprises at least one of an ultrasound device and a magnetic resonance imaging device.

63. The system as in claim 56, wherein the monitoring apparatus comprises a bioluminescence imaging system.

64. The system as in claim 56, further comprising a management system for controlling the delivery of the therapeutic beam according to monitoring information in real time.

65. The system as in claim 56, further comprising a user interface that provides at least one of:
a visual representation of the therapeutic beam in relation to the tissue;
an image that varies in at least one of brightness, color and/or numerical value according to a quantity of free radicals present in the tissue;
at least one image as an overlay to an image of the therapeutic beam; and,
an image of the tissue.

66. The system as in claim 56, wherein the tissue comprises at least one of an organ, a tumor, and tissue surrounding a tumor.

67. A method for providing radiation therapy, the method comprising:
determining a treatment plan for delivering radiation to a tissue of a patient;
selecting a radiation therapy system that comprises a therapeutic apparatus configured for delivery of a therapeutic beam of radiation to tissue of a patient, a monitoring apparatus configured to monitor production of biomarkers within the tissue during the delivery of the therapeutic beam, and an apparatus for delivery of a secondary beam of microwave radiation, the biomarkers comprising at least one of free radicals, burst vesicles, dissociation of an adduct and an indication of protein damage;
delivering the secondary beam along a trajectory of the therapeutic beam to pre-align the therapeutic beam; and
delivering the therapeutic beam of radiation to the patient with the radiation therapy system.

68. The method as in claim 67, further comprising monitoring the production of free radicals, wherein the monitoring comprises quantifying the free radicals, and wherein the quantifying comprises determining a signal intensity of a proton-electron double-resonance imaging (PEDRI) signal.

69. The method as in claim 67, further comprising monitoring the production of free radicals, wherein the monitoring further comprises observing oxygenation levels within the tissue.

70. The method as in claim 67, further comprising monitoring an indication from a contrast agent wherein the contrast agent comprises at least one of a dispersion of nanoparticles and a surfactant-dye aggregate.

71. The method as in claim 67, further comprising monitoring movement of an organ within the tissue and providing a graphic output of the movement.

72. The method as in claim 67, further comprising providing a graphic output of the trajectory of the therapeutic beam.

73. The method as in claim 67, further comprising adjusting the delivering of the therapeutic beam according to a comparison of monitoring information and the treatment plan.

75. The method as in claim 67, further comprising at least one of monitoring the production of the free radicals and adjusting the delivering of the therapeutic beam in substantially real-time.

75. A computer program product for controlling radiation therapy, the computer program product comprising machine executable instructions stored on machine readable media, the product comprising instructions for:
delivering a secondary beam of microwave radiation along a trajectory of a therapeutic beam of radiation to pre-align the therapeutic beam;
delivering the therapeutic beam along the trajectory after pre-alignment;
receiving information descriptive of production of free radicals within tissue of a patient during radiation therapy, the free radicals resulting from the therapeutic beam;
comparing the production to a treatment plan; and
controlling the radiation therapy according to results of the comparison.

76. The computer program product as in claim 75, further comprising instructions for performing the receiving, comparing and controlling in real-time.
